(19) United States
(12) Patent Application Publication
(10) Pub. No.: US 2022/0379311 A1
(43) Pub. Date: Dec. 1, 2022

(54) CELL PURIFICATION MODULE, CELL PURIFICATION SYSTEM AND OPERATION METHOD THEREOF

(71) Applicant: Industrial Technology Research Institute, Hsinchu (TW)

(72) Inventors: Lih-Tao Hsu, Taoyuan City (TW); Shen-Hua Peng, Taoyuan City (TW); Cheng-Yi Wu, Hsinchu County (TW); Jeng-Liang Kuo, Hsinchu City (TW); Meng-Hsueh Lin, Taoyuan City (TW); Chih-Chieh Huang, Miaoli County (TW); Wei-Lin Yu, Hsinchu County (TW); Hui-Ting Huang, Hsinchu County (TW)

(73) Assignee: Industrial Technology Research Institute, Hsinchu (TW)

(21) Appl. No.: 17/824,929

(22) Filed: May 26, 2022

Related U.S. Application Data
(60) Provisional application No. 63/194,186, filed on May 28, 2021.

(30) Foreign Application Priority Data
May 28, 2021 (TW) 110119482

(51) Int. Cl. B01L 3/00

(52) U.S. Cl. CPC B01L 3/502761; B01L 2200/0652; B01L 2400/043; B01L 2300/0832; B01L 2300/0681

(57) ABSTRACT

A cell purification module, configured to purify multiple cells from a fluid sample is provided. The cell purification module includes a hollow column, multiple hollow fiber membranes, at least one first magnetic component, a fluid sample inlet end, and a fluid sample outlet end. The hollow column has a first opening, a second opening, and an accommodating space connecting the first opening and the second opening. The hollow fiber membranes are disposed in the accommodating space and each hollow fiber membrane has multiple pores. The first magnetic component is disposed at a periphery of the hollow column. The fluid sample inlet end and the fluid sample outlet end are respectively disposed at two ends of the hollow column. The hollow fiber membranes extend in an axial direction of the hollow column, and are arranged in a radial direction of the hollow column. A cell purification system is also provided.

### CROSS-REFERENCE TO RELATED APPLICATION

This application claims the priority benefit of U.S. provisional application Ser. No. 63/194,186, filed on May 28, 2021, and Taiwan application serial no. 110119482, filed on May 28, 2021. The entirety of each of the abovementioned patent applications is hereby incorporated by reference herein and made a part of this specification.

### TECHNICAL FIELD

This disclosure relates to a purification module and a purification system, and in particular to a cell purification module and a cell purification system capable of reducing contamination, simplifying a process, saving time, or improving purification efficiency.

### BACKGROUND

In general, in order to purify cellular fluid sample containing magnetic beads and extracellular substances, the purification method is performed by firstly to use a magnetic bead removal device to remove the magnetic beads in the cellular fluid and secondly transfer the cellular fluid (after the removal of the magnetic beads) to a cell concentration device to carry out cell concentration, washing (removal of extracellular substances), and purification.

However, the process of transferring the cellular fluid sample between the two devices may increase a risk of contamination. In addition, using the magnetic bead removal device first to remove the magnetic beads, and then using the cell concentration device to perform cell purification may also make the overall purification process more complicated and time-consuming.

Therefore, there is an urgent need to develop a device to simplify the purification process, save time, and improve purification efficiency simultaneously.

### SUMMARY

This disclosure provides a cell purification module, a cell purification system and an operation method thereof, which are capable of reducing contamination, simplifying purification process, saving time, and improving purification efficiency.

The cell purification module of the disclosure is configured to purify multiple cells from a fluid sample. The cell purification module includes a hollow column, multiple hollow fiber membranes, at least one first magnetic component, a fluid sample inlet end, and a fluid sample outlet end. The hollow column has a first opening, a second opening opposite to the first opening, and an accommodating space connecting the first opening and the second opening. The multiple hollow fiber membranes are disposed in the accommodating space and each of the hollow fiber membranes has multiple pores. The first magnetic component is disposed at a periphery of the hollow column. The fluid sample inlet end is disposed at one end of the hollow column. The fluid sample outlet end is disposed at another end of the hollow column. The hollow column has an axial direction extending in a direction of an axis of the hollow column, a radial direction extending in a cross-sectional radius direction of the hollow column and perpendicular to the axial direction of the hollow column, and a circumferential direction surrounding the axis of the hollow column. The multiple hollow fiber membranes extend in the axial direction of the hollow column, and the multiple hollow fiber membranes are arranged in the radial direction of the hollow column.

In an embodiment of the disclosure, the at least one first magnetic component includes one or more first magnetic components, which are disposed at at least one side along the circumferential direction of the hollow column.

In an embodiment of the disclosure, the multiple first magnetic components include one or more first magnetic components annularly surrounding the hollow column.

In an embodiment of the disclosure, the cell purification module further includes at least one filtrate outlet end. The filtrate outlet end is disposed on the hollow column, so that a filtrate flowing out from the multiple pores flows out through the filtrate outlet end.

In an embodiment of the disclosure, the fluid sample includes at least one cellular fluid and multiple magnetic beads. The cellular fluid includes at least multiple cells and a cell culture medium.

In an embodiment of the disclosure, the cell purification module further includes multiple blind tubes disposed in the accommodating space. The multiple blind tubes extend in the axial direction of the hollow column and are arranged in the radial direction of the hollow column. The multiple blind tubes are surrounded by the multiple hollow fiber membranes.

In an embodiment of the disclosure, the multiple blind tubes and the multiple hollow fiber membranes are all annularly arranged around the axis of the hollow column. A ratio of a total length of the multiple blind tubes in the radial direction of the hollow column to a diameter of the hollow column is 1:10 to 8:10.

In an embodiment of the disclosure, a size of the multiple cells is larger than a pore diameter of the multiple pores of the multiple hollow fiber membranes.

The disclosure provides a cell purification system including the aforementioned cell purification module, a storage container, and a peristaltic pump. The storage container is configured to store the fluid sample. The storage container is connected to the fluid sample outlet end of the cell purification module through a first pipeline. The peristaltic pump is configured to push the fluid sample to flow. The peristaltic pump is respectively connected to the storage container and the fluid sample inlet end of the cell purification module through a second pipeline and a third pipeline.

In an embodiment of the disclosure, the aforementioned cell purification system further includes at least one second magnetic component. The second magnetic component is disposed at a periphery of the third pipeline and is adjacent to the fluid sample inlet end.

In an embodiment of the disclosure, the at least one second magnetic component includes one or more second magnetic components, which are respectively disposed at at least one side of the third pipeline.

In an embodiment of the disclosure, the at least one second magnetic component includes one or more second magnetic components, which annularly surround the third pipeline.

The disclosure provides an operation method of a cell purification system, which includes following steps. In Step (a), the aforementioned cell purification system is provided. In Step (b), the peristaltic pump is used to output the fluid sample in the storage container to the peristaltic pump through the second pipeline. In Step (c), the peristaltic pump is used to input the fluid sample into the cell purification module through the third pipeline and the fluid sample inlet end. In Step (d), the peristaltic pump is used to enable a filtrate to flow out through the at least one filtrate outlet end, so as to concentrate the fluid sample and output the concentrated fluid sample through the fluid sample outlet end. In Step (e), the peristaltic pump is used to re-input the concentrated fluid sample from the cell purification module from Step (d) into the storage container through the first pipeline. In Step (f), the Steps (b) to (e) are repeated.

In an embodiment of the disclosure, the operation method of the cell purification system further includes adding a washing solution before performing the Step (f).

Based on the above description, in the cell purification module, the cell purification system, and the operation method thereof according to the embodiments of the disclosure, the cell culture medium and the extracellular substances in the cellular fluid may be discharged through the disposition of the pores of the hollow fiber membranes and the filtrate outlet end, so that the cellular fluid may be concentrated and purified. The multiple magnetic beads in the cellular fluid may be adsorbed on the inner walls of the hollow fiber membranes through the disposition of the first magnetic component to remove the magnetic beads, so that the magnetic beads in the cellular fluid may be removed. In comparison to the related art in which the magnetic bead removal device is used before the cell concentration device (i.e., the magnetic beads are removed first, and then the cells are concentrated), the cell purification module and cell purification system according to the embodiments are enclosed systems that may remove the magnetic beads and concentrate the cells concurrently. Therefore, the described enclosed purification system can provide the effects of reducing contamination, simplifying purification process, saving time, and improving purification efficiency.

To make the aforementioned more comprehensible, several embodiments accompanied with drawings are described in detail as follows.

### BRIEF DESCRIPTION OF THE DRAWINGS

FIG. 1A is a three-dimensional schematic view of a cell purification module according to an embodiment of the disclosure.

FIG. 1B is a schematic view of a structure of the cell purification module in FIG. 1A.

FIG. 1C is a schematic cross-sectional view of the cell purification module in FIG. 1B.

FIG. 1D is an enlarged view of an area A of the cell purification module in FIG. 1B.

FIGS. 2A to 2B are schematic structural view of cell purification modules according to multiple embodiments of the disclosure.

FIG. 3A is a schematic structural view of a cell purification module according to an embodiment of the disclosure.

FIG. 3B is a schematic cross-sectional view of a cell purification module according to an embodiment of the disclosure.

FIG. 4 is a schematic structural view of a cell purification system according to an embodiment of the disclosure.

FIG. 5 is a schematic partial structural view of a cell purification system according to another embodiment of the disclosure.

FIGS. 6A to 6C are results of using cell purification systems including the cell purification modules in FIGS. 2A and 2B and the cell purification system in FIG. 5 to remove the magnetic beads.

FIG. 7 shows proportions of the cells containing specific cell markers in a fluid sample before and after purification.

### DESCRIPTION OF THE EMBODIMENTS

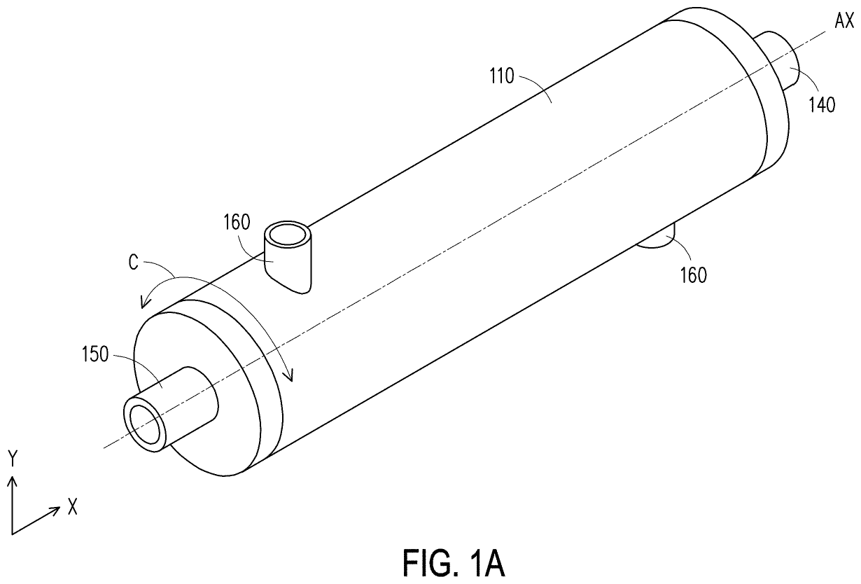

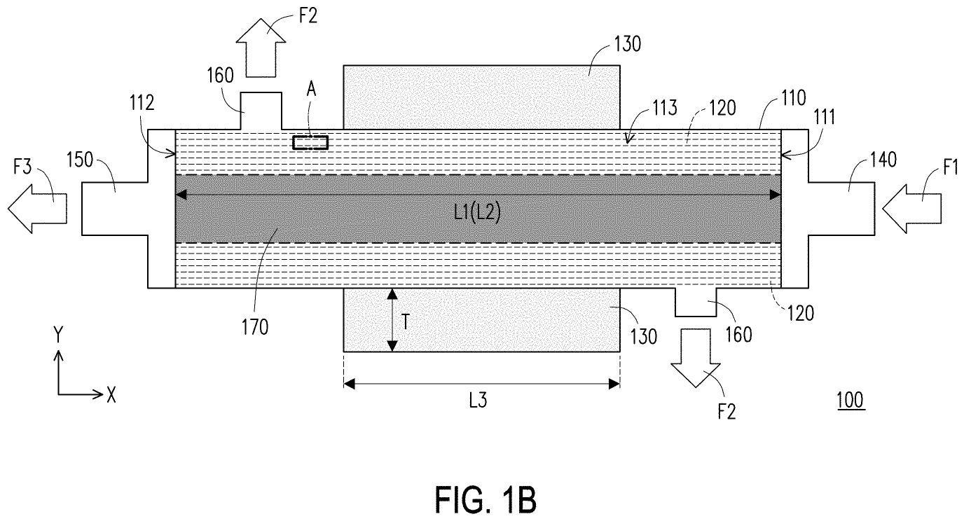

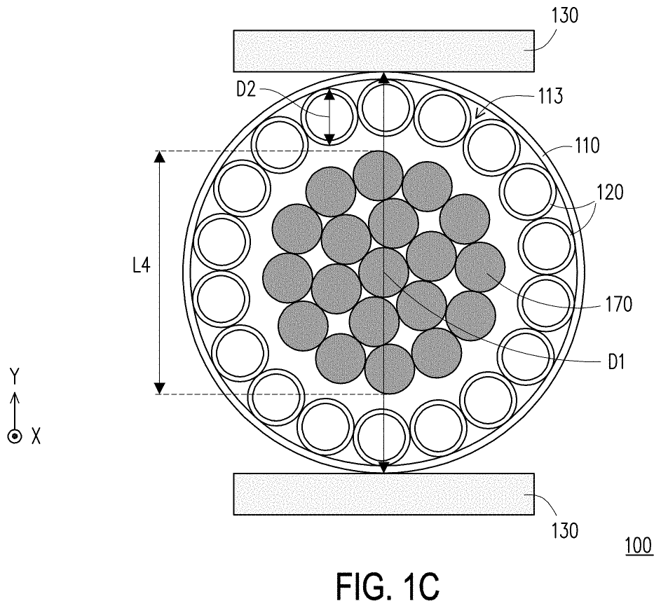

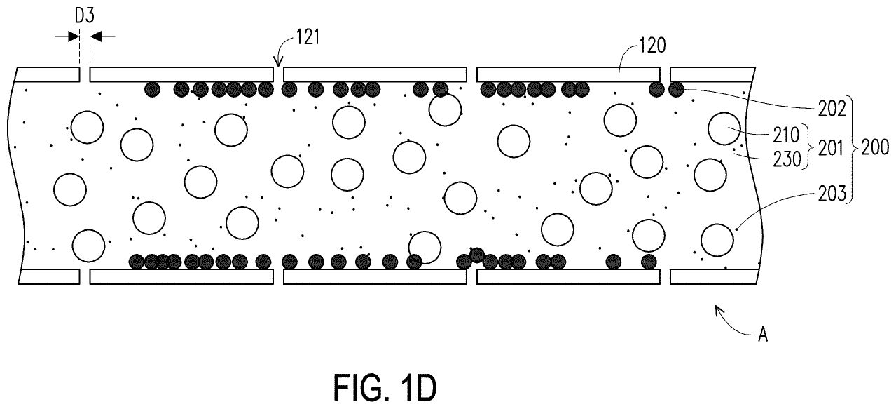

FIG. 1A is a three-dimensional schematic view of a cell purification module according to an embodiment of the disclosure. FIG. 1B is a schematic view of a structure of the cell purification module in FIG. 1A. FIG. 1C is a schematic cross-sectional view of the cell purification module in FIG. 1B. FIG. 1D is an enlarged view of an area A of the cell purification module in FIG. 1B. A cell purification module 100 may be used to purify multiple cells 210 from a fluid sample 200. In addition, FIG. 1C omits illustrations of a fluid sample inlet end 140, a fluid sample outlet end 150, and a filtrate outlet end 160 for clarity.

Referring to FIGS. 1B to 1D, the cell purification module 100 includes a hollow column 110, multiple hollow fiber membranes 120, at least one first magnetic component 130, the fluid sample inlet end 140, the fluid sample outlet end 150, at least one filtrate outlet end 160, and multiple blind tubes 170. The hollow column 110 has a first opening 111, a second opening 112 opposite to the first opening 111, and an accommodating space 113 connecting the first opening 111 and the second opening 112. The hollow column 110 may be, for example, a hollow cylinder or a hollow columnar structure of other shapes, but the disclosure is not limited thereto. The hollow column 110 also has an axial direction X extending in a direction of an axis of the hollow column, a radial direction Y extending in a cross-sectional radius direction of the hollow column 110 and perpendicular to the axial direction of the hollow column 110, and a circumferential direction C surrounding the axis AX of the hollow column 110. A material of the hollow column 110 is, for example, polyurethane, but the disclosure is not limited thereto.

In an embodiment, a diameter D1 of the hollow column 110 may be 9 mm to 150 mm, for example, about 10 mm to 130 mm, about 20 mm to 120 mm, about 30 mm to 110 mm, about 40 mm to 100 mm, about 50 mm to 90 mm, about 15 mm, about 25 mm, about 45 mm, about 65 mm, about 85 mm, about 105 mm, about 125 mm, etc., but the disclosure is not limited thereto. A length L1 of the hollow column 110 may be 120 mm to 1200 mm, for example, about 130 mm to 1100 mm, about 140 mm to 1000 mm, about 150 mm to 900 mm, about 160 mm to 800 mm, and about 170 mm to 700 mm, about 200 mm to 600 mm, about 240 mm, about 360 mm, about 480 mm, about 650 mm, about 760 mm, about 850 mm, about 960 mm, about 1050 mm, about 1150 mm, etc., but the disclosure is not limited thereto. In some embodiments, the diameter D1 and the length L1 of the hollow column 110 may also be adjusted as needed.

In the embodiment, the fluid sample 200 may include a cellular fluid 201 and multiple magnetic beads 202. The cellular fluid 201 may include the multiple cells 210 and a cell culture medium 230. The cell culture medium 230 may include extracellular substances 203, for example, may include growth factors or animal serum added during a cell culture process, enzymes (such as trypsin) used during a manufacturing process, human serum albumin (HSA), cell metabolites, and other components that are not to be recovered, but the disclosure is not limited thereto.

In the embodiment, the multiple hollow fiber membranes 120 are disposed in the accommodating space 113. The multiple hollow fiber membranes 120 extend in the axial direction X of the hollow column 110, and the multiple hollow fiber membranes 120 are arranged in the radial direction Y of the hollow column 110. In the embodiment, a length L2 of the hollow fiber membrane 120 may be substantially equal to the length L1 of the hollow column 110, but the disclosure is not limited thereto. The length L2 of the hollow fiber membrane 120 may be 120 mm to 1100 mm, for example, about 130 mm to 1000 mm, about 140 mm to 900 mm, about 150 mm to 800 mm, about 160 mm to 700 mm, about 170 mm to 600 mm, about 135 mm, about 145 mm, about 155 mm, about 165 mm, about 185 mm, about 205 mm, about 225 mm, about 275 mm, about 325 mm, about 375 mm, about 450 mm, about 550 mm, about 650 mm, about 750 mm, about 850 mm, about 950 mm, about 1050 mm, etc., but the disclosure is not limited thereto. A diameter D2 of the hollow fiber membrane 120 may be 0.175 mm to 1.75 mm, for example, about 0.18 mm to 1.6 mm, about 0.19 mm to 1.5 mm, about 0.2 mm to 1.4 mm, about 0.21 mm to 1.3 mm, about 0.22 mm to 1.2 mm, about 0.185 mm, about 0.195 mm, about 0.205 mm, about 0.215 mm, about 0.225 mm, about 0.3 mm, about 0.4 mm, about 0.6 mm, about 0.8 mm, about 1.0 mm, about 1.25 mm, about 1.45 mm, about 1.65 mm, etc., but the disclosure is not limited thereto. In some embodiments, the length L2 and the diameter D2 of the hollow fiber membrane 120 may also be adjusted as needed. In the embodiment, a material of the hollow fiber membrane 120 is, for example, polysulfone, but the disclosure is not limited thereto.

In the embodiment, each of the hollow fiber membranes 120 has multiple pores 121 on a tube wall. A pore size D3 of the pore 121 may be 0.2 μm to 1 μm, for example, about 0.3 μm to 0.9 μm, about 0.4 μm to 0.8 μm, about 0.5 μm to 0.7 μm, about 0.35 μm, about 0.45 μm, about 0.55 μm, about 0.65 μm, about 0.75 μm, about 0.85 μm, about 0.95 μm, etc., but the disclosure is not limited thereto. In some embodiments, the pore size D3 of the pore 121 may also be adjusted as needed. In the embodiment, since a size of a cell 210 and a size of the magnetic bead 202 may both be larger than the pore size D3 of the pore 121 of the hollow fiber membrane 120, the cells 210 and the magnetic beads 202 will not flow into the accommodating space 113 through the pores 121. However, the cell culture medium 230 and the extracellular substances 203 in the fluid sample 200 may flow into the accommodating space 113 through the pores 121 of the hollow fiber membrane 120.

In an embodiment, the first magnetic component 130 is disposed at the periphery of the hollow column 110, so that the multiple magnetic beads 202 in the fluid sample 200 may be absorbed on an inner wall of the hollow fiber membrane 120 through a magnetic force of the first magnetic component 130 to remove the magnetic beads 202, as shown in FIG. 1D. To be specific, in an embodiment, FIGS. 1B and 1C schematically illustrate two first magnetic components 130. The two first magnetic components 130 are respectively disposed at two sides of the hollow column 110. The two first magnetic components 130 are, for example, directly or indirectly bound to two sides along the circumferential direction C of the hollow column 110, but the disclosure is not limited thereto. The two first magnetic components 130 are, for example, arranged on upper and lower sides of the hollow column 110 in a direction Y perpendicular to the extending direction X of the hollow column 110, but the disclosure is not limited thereto. The two first magnetic components 130 are, for example, disposed at a distance of about ½ between the fluid sample inlet end 140 and the fluid sample outlet end 150, but the disclosure is not limited thereto. In the embodiment, a material of the first magnetic component 130 is, for example, neodymium iron boron magnet (Nd—Fe—B), but the disclosure is not limited thereto.

In the embodiment, a thickness T of the first magnetic component 130 may be 3 mm to 15 mm, for example, about 4 mm to 14 mm, about 5 mm to 13 mm, about 6 mm to 12 mm, about 7 mm to 11 mm, about 8 mm to 10 mm, about 3.5 mm, about 4.5 mm, about 5.5 mm, about 6.5 mm, about 7.5 mm, about 8.5 mm, about 9.5 mm, etc., but the disclosure is not limited thereto. A length L3 of the first magnetic component 130 may be 30 mm to 40 mm, for example, about 31 mm to 39 mm, about 32 mm to 38 mm, about 33 mm to 37 mm, about 34 mm to 36 mm, about 31.5 mm, about 32.5 mm, about 33.5 mm, about 34.5 mm, about 35.5 mm, about 36.5 mm, about 37.5 mm, etc., but the disclosure is not limited thereto. A ratio of the thickness T of the first magnetic component 130 to the diameter D1 of the hollow column 110 may be about 1.2:1, about 1.3:1, about 1.4:1, about 1.5:1, about 1.6:1, about 1.7:1, about 1.8:1, about 2:1, etc., but the disclosure is not limited thereto. A ratio of the length L3 of the first magnetic component 130 to the length L1 of the hollow column 110 may be 2:1 to 4:1, for example, about 2.2:1, about 2.5:1, about 2.8:1, about 3:1, about 3.3:1, about 3.5:1, about 3.75:1, etc., but the disclosure is not limited thereto. In some embodiments, the length L3 of the first magnetic component 130 may also be substantially equal to the length L1 of the hollow column 110 (not shown). In some embodiments, the thickness T and the length L3 of the first magnetic component 130 may also be adjusted as needed.

In the embodiment, the fluid sample inlet end 140 is disposed at one end of the hollow column 110, and the fluid sample outlet end 150 is disposed at another end of the hollow column 110. In the extending direction X of the hollow column 110, the fluid sample inlet end 140 and the fluid sample outlet end 150 are respectively disposed at two opposite ends of the hollow column 110. To be specific, the fluid sample inlet end 140 may be connected to the first opening 111 of the hollow column 110, so that the fluid sample 200 may flow into the hollow fiber membranes 120 through the fluid sample inlet end 140. The fluid sample outlet end 150 may be connected to the second opening 112 of the hollow column 110, so that the fluid sample 200 or the concentrated fluid sample 200 located in the hollow fiber membranes 120 may flow out through the fluid sample outlet end 150. In other words, according to a fluid sample inflow direction F1 and a fluid sample outflow direction F3, the fluid sample 200 may flow into the hollow fiber membranes 120 from the fluid sample inlet end 140 and flow out from the fluid sample outlet end 150.

In the embodiment, the filtrate outlet end 160 is disposed on the hollow column 110, and the filtrate outlet end 160 may communicate with the accommodating space 113 of the hollow column 110, so that the filtrate flowing out from the pores 121 of the hollow fiber membranes 120 may flow out through the filtrate outlet end 160. Namely, according to a filtrate outflow direction F2, the filtrate may flow out from the filtrate outlet end 160. The filtrate is located in the accommodating space 113 of the hollow column 110. The filtrate includes the cell culture medium 230 and the extracellular substances 203 that flow through the pores 121 and flow to the accommodating space 113. In the embodiment, FIG. 1B schematically illustrates two filtrate outlet ends 160, which are respectively adjacent to the fluid sample inlet end 140 and the fluid sample outlet end 150, but the disclosure is not limited thereto.

In the embodiment, multiple blind tubes 170 are disposed in the accommodating space 113. The multiple blind tubes 170 extend in the axial direction X of the hollow column 110, and the multiple blind tubes 170 are arranged in the radial direction Y of the hollow column 110. The blind tube 170 may be, for example, a solid cylinder or a hollow tubular structure with closed inlet and outlet, but the disclosure is not limited thereto. In the schematic side view of the cell purification module 100 of the embodiment (as shown in FIG. 1C), the multiple blind tubes 170 may be surrounded by the multiple hollow fiber membranes 120, so that the multiple blind tubes 170 are disposed in the center of the accommodating space 113. In the embodiment, by disposing the multiple blind tubes 170 in the center of the accommodating space 113, the hollow fiber membranes 120 surrounding the multiple blind tubes 170 may be closer to the first magnetic component 130, so that the hollow fiber membranes 120 may be located within a range covered by the magnetic force of the first magnetic component 130, thereby increasing the removal of the magnetic beads 202. In the embodiment, the blind tubes 170 may also be used to support the hollow fiber membranes 120 to fix the hollow fiber membranes 120 in the hollow column 110. A material of the blind tube 170 is, for example, polyurethane or polysulfone, but the disclosure is not limited thereto. In some embodiments, the material of the blind tube 170 may also include metal or magnets to improve the adsorption of the magnetic beads 202 on the inner walls of the hollow fiber membranes 120 and the removal of the magnetic beads 202. For example, the metal includes iron, aluminum, nickel, cobalt, or a combination thereof. Magnets include neodymium iron boron magnets (Nd—Fe—B) and samarium-cobalt magnets.

Moreover, in the embodiment, the multiple blind tubes 170 and the multiple hollow fiber membranes 120 are all arranged annularly around the axis AX of the hollow column 110. A ratio of a total length L4 of the multiple blind tubes 170 in the radial direction Y of the hollow column 110 to the diameter D1 of the hollow column 110 may be 1:10 to 8:10, for example, about 2:10, about 3:10, about 4:10, about 5:10, about 6:10, about 7:10, etc., but the disclosure is not limited thereto. Since the ratio of the total length L4 of the blind tubes 170 in the radial direction Y of the hollow column 110 to the diameter D1 of the hollow column 110 is about 1:10 to 8:10, it is ensured that all of the hollow fiber membranes 120 are located within the range covered by the magnetic force of the first magnetic component 130, thereby improving the removal of the magnetic beads 202.

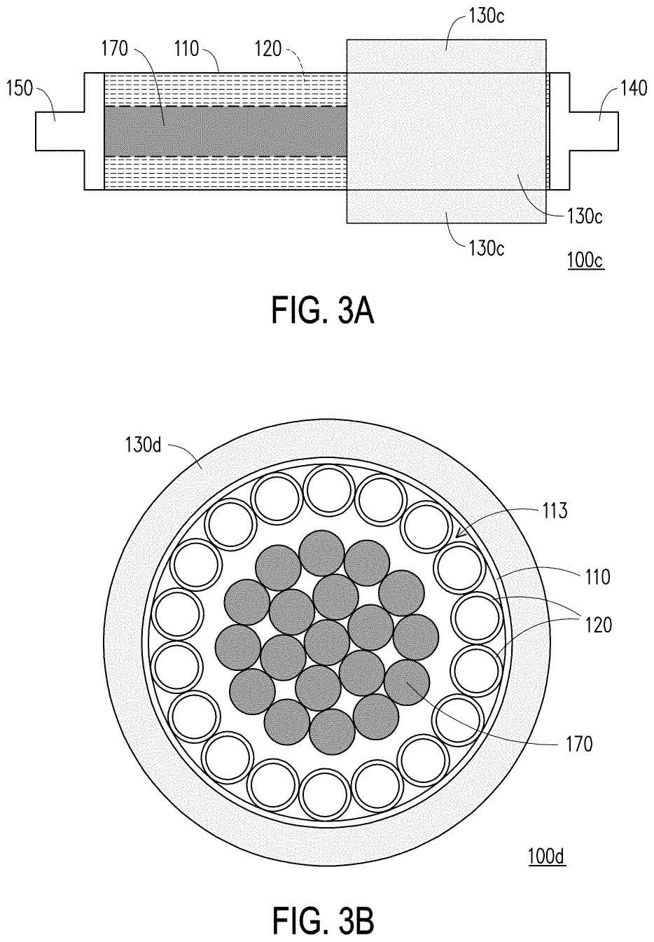

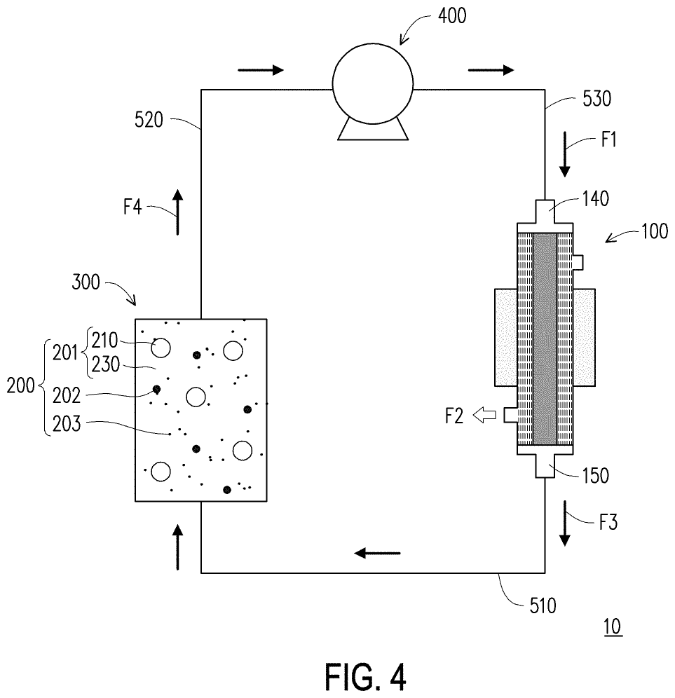

Although the number of the first magnetic components 130 schematically illustrated in the embodiment is two, and the two first magnetic components 130 are respectively disposed at two sides of the hollow column 110, the disclosure does not limit the number and configuration positions of the first magnetic components, as long as the first magnetic components 130 may be disposed at the periphery of the hollow column 110. In some embodiments, the number of the first magnetic component may also be one, which is disposed at one side of the hollow column (not shown). In some embodiments, the number of the first magnetic components may also be two or more, which are respectively disposed at two or more sides of the hollow column (not shown). In some embodiments, the number of the first magnetic components may also be two or more, which annularly surround the hollow column (as shown in FIG. 3A).

Although the number of the blind tubes 170 schematically illustrated in the embodiment as plural, the disclosure does not limit the number of the blind tubes, as long as the hollow fiber membranes 120 may be located within the range of the magnetic force of the first magnetic components 130. In some embodiments, the number of the blind tube may also be one (not shown). In some embodiments, the blind tubes may not be additionally configured.

In brief, in the cell purification module 100 of the embodiment, since the cell culture medium 230 and the extracellular substances 203 in the fluid sample 200 may pass through the pores 121 of the hollow fiber membranes 120 and the filtrate outlet end 160 to flow out, and the multiple magnetic beads 202 in the fluid sample 200 may be absorbed on the inner walls of the hollow fiber membranes 120 through the magnetic force of the first magnetic components 130, when the fluid sample 200 flows through the cell purification module 100, the fluid sample 200 may be concentrated and the magnetic beads 202 in the fluid sample 200 may be removed, so as to achieve the effect of purifying the cells 210 from the fluid sample 200. Therefore, compared to the related art of first using the magnetic bead removal device and then using the cell concentration device (i.e., the magnetic beads are removed first, and then the cells are concentrated), the embodiment only needs to use the cell purification module 100 to concurrently remove the magnetic beads 202 and concentrate the cells 210, so that the cell purification module 100 of the embodiment may simplify the process, save time, or improve purification efficiency.

Other embodiments are further described below. It should be noted that reference numerals of the elements and a part of contents of the aforementioned embodiment are also used in the following embodiment. The same reference numerals denote the same or similar elements, and descriptions of the same technical contents are omitted. Reference may be made to the aforementioned embodiment for descriptions of the omitted parts, which are not repeated in the following embodiment.

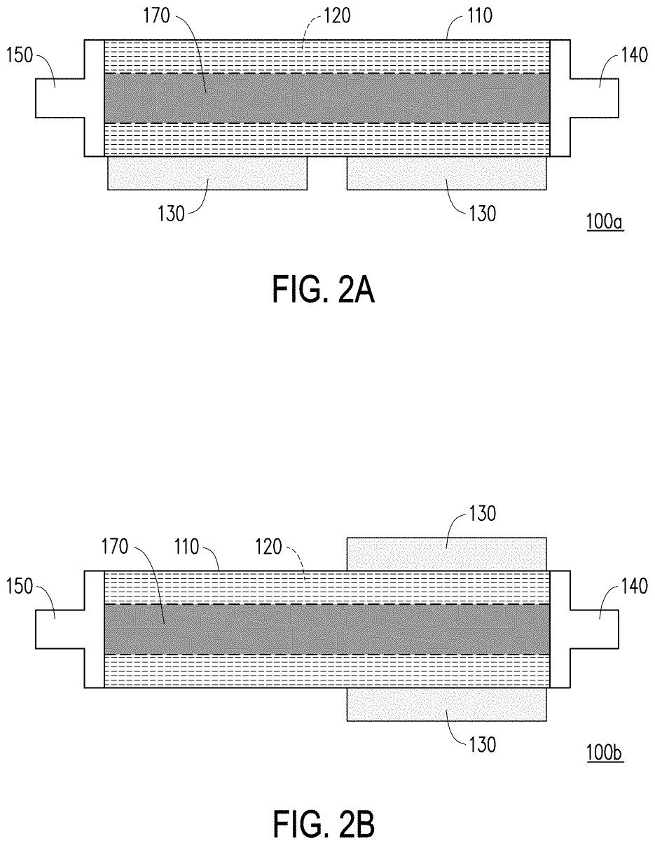

FIGS. 2A and 2B are schematic structural views of cell purification modules according to multiple embodiments of the disclosure. The illustration of the filtrate outlet end 160 is omitted in FIGS. 2A and 2B for clarity of illustration and description.

First, referring to FIGS. 1B and 2A concurrently, a cell purification module 100a in the embodiment is similar to the cell purification module 100 in FIG. 1B, and a main difference therebetween is that in the purification module 100a of the embodiment, the two first magnetic components 130 are both disposed at the same side of the hollow column 110. The length of the hollow column 110 is 160 mm and the diameter thereof is 10 mm. The total length of the blind tubes 170 in the radial direction of the hollow column 110 is 5 mm. The thickness of the first magnetic component 130 is 15 mm and the length thereof is 40 mm.

Then, referring to FIGS. 1B and 2B concurrently, a cell purification module 100b in the embodiment is similar to the cell purification module 100 in FIG. 1B, and the main difference therebetween is that in the purification module 100b of the embodiment, the two first magnetic components 130 are both adjacent to the fluid sample inlet end 140 and far away from the fluid sample outlet end 150. The length of the hollow column 110 is 160 mm and the diameter thereof is 10 mm. The total length of the blind tubes 170 in the radial direction of the hollow column 110 is 5 mm. The thickness of the first magnetic component 130 is 15 mm and the length thereof is 40 mm.

FIG. 3A is a schematic structural view of a cell purification module according to an embodiment of the disclosure. Referring to FIGS. 1B, 1C and 3A concurrently, a cell purification module 100c of the embodiment is similar to the cell purification module 100 in FIGS. 1B and 1C, and the main difference therebetween is that in the cell purification module 100c of the embodiment, the number of the first magnetic components 130c is four, and the first magnetic components 130c are adjacent to the fluid sample inlet end 140 and far away from the fluid sample outlet end 150. The four first magnetic components 130c are annularly configured around the hollow column 110, so that the four first magnetic components 130c may annularly surround the hollow column 110. The hollow column 110 has a length of 160 mm and a diameter of 10 mm. The total length of the blind tubes 170 in the radial direction of the hollow column 110 is 5 mm. The thickness of the first magnetic component 130 is 3 mm and the length thereof is 30 mm.

FIG. 3B is a schematic cross-sectional view of a cell purification module according to an embodiment of the disclosure. Referring to FIGS. 1C and 3B concurrently, a cell purification module 100d of the embodiment is similar to the cell purification module 100 in FIG. 1C, and a main difference therebetween is that in the cell purification module 100d of the embodiment, the number of the first magnetic component 130d is one, and the first magnetic component 130d is embodied as a ring structure, so that the first magnetic component 130d may annularly surround the hollow column 110.

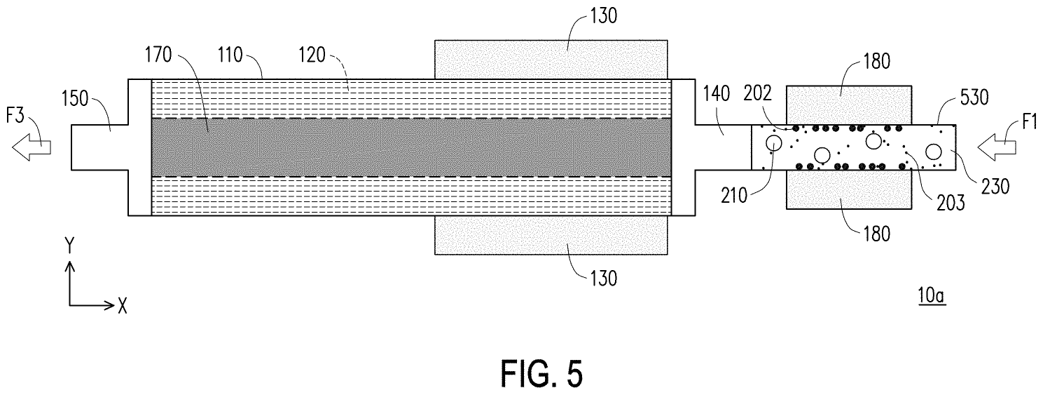

FIG. 4 is a schematic structural view of a cell purification system according to an embodiment of the disclosure. Referring to FIG. 4, a cell purification system 10 of the embodiment includes the aforementioned cell purification module 100, a storage container 300, a peristaltic pump 400, a first pipeline 510, a second pipeline 520, and a third pipeline 530.

To be specific, the storage container 300 may be used to store the fluid sample 200. The storage container 300 may be connected to the peristaltic pump 400 through the second pipeline 520, and the storage container 300 may be connected to the fluid sample outlet end 150 of the cell purification module 100 through the first pipeline 510.

The peristaltic pump 400 may be used to push the fluid sample 200 to flow. The peristaltic pump 400 is respectively connected to the storage container 300 and the fluid sample inlet end 140 of the cell purification module 100 through the second pipeline 520 and the third pipeline 530.

To be more specific, the cell purification system 10 of the embodiment is a circulatory system, and the peristaltic pump 400 is used to push the fluid sample 200, so that the fluid sample 200 may circulate and flow according to a sequence as follows. The storage container 300→the second pipeline 520→the peristaltic pump 400→the third pipeline 530→the cell purification module 100→the first pipeline 510→the storage container 300.

An operation method of the cell purification system of the embodiment may include following steps. In Step (a), the cell purification system is provided. In Step (b), the peristaltic pump 400 is used to output the fluid sample 200 in the storage container 300 to the peristaltic pump 400 through the second pipeline 520. In Step (c), the peristaltic pump 400 is used to input the fluid sample 200 into the cell purification module 100 through the third pipeline 530. In Step (d), the peristaltic pump 400 is used to enable the filtrate to flow out through the filtrate outlet end 160, so as to concentrate the fluid sample 200 and output the concentrated fluid sample 200 through the fluid sample outlet end 150. In Step (e), the peristaltic pump 400 is used to re-input the concentrated fluid sample 200 outputted from the cell purification module 100 into the storage container 300 through the first pipeline 510. In Step (f), the Steps (b) to (e) are repeated. The fluid sample 200 flows out of the storage container 300 according to a fluid sample flow direction F4 and enters the peristaltic pump 400. The fluid sample 200 flows out of the peristaltic pump 400 according to the fluid sample inflow direction F1 and enters the cell purification module 100. The filtrate flows out from the cell purification module 100 according to the filtrate outflow direction F2, and the fluid sample 200 flows out from the cell purification module 100 according to the fluid sample outflow direction F3 and enters the storage container 300.

In some embodiments, the operation method of the above cell purification system further includes the following step. A washing solution is added before performing the Step (0. The washing solution is, for example, phosphate buffered saline (PBS), but the disclosure is not limited thereto.

In addition, in the embodiment, a processing volume of the cell purification module 100 is about 100 milliliters to 100 liters of the fluid sample 200, and a processing time of the fluid sample 200 of 2 liters is about 40 minutes, but the disclosure is not limited thereto. In some embodiments, when a specification of the cell purification module is adjusted as needed, the processing volume and processing time of the adjusted cell purification module may also be adjusted accordingly.

In the embodiment, since the cell purification system 10 is a closed system, and the cell purification system 10 may concurrently remove the magnetic beads 202 and concentrate the cells 210, compared to the related art in which the magnetic bead removal device is first used before switching to the cell concentration device (i.e., the magnetic beads are removed first, and then the cells are concentrated), the cell purification system 10 of the embodiment does not have the step of switching between different devices, thereby decreasing a risk of contamination.

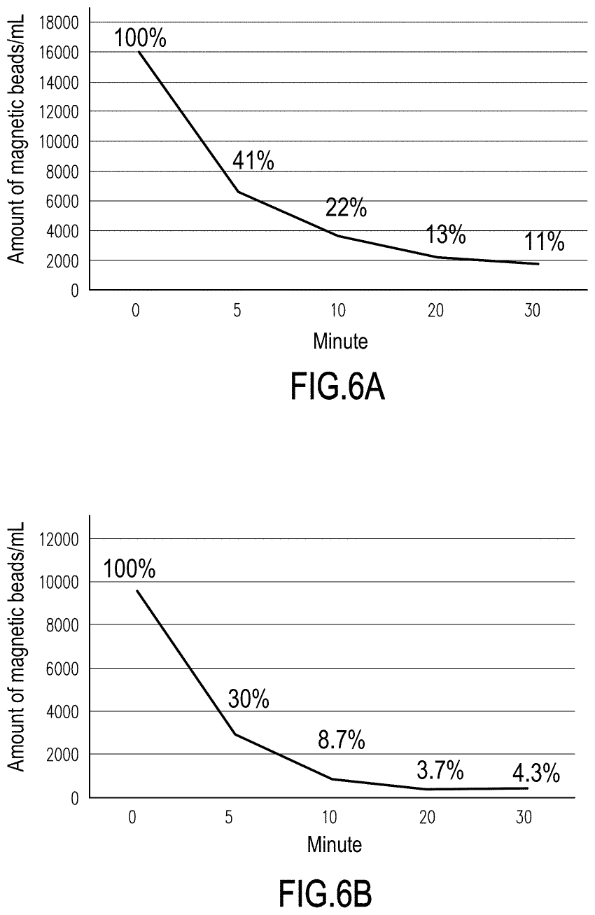

FIG. 5 is a schematic partial structural view of a cell purification system according to another embodiment of the disclosure. Referring to FIGS. 4 and 5 concurrently, a cell purification system 10a of the embodiment is similar to the cell purification system 10 in FIG. 4, and a main difference therebetween is that the cell purification system 10a of the embodiment further includes two second magnetic components 180 respectively disposed at two sides of the hollow column 110. The second magnetic components 180 are disposed at a periphery of the third pipeline 530 and adjacent to the fluid sample inlet end 140 of the cell purification module 100, so that the multiple magnetic beads 202 in the fluid sample 200 may be adsorbed on the inner wall of the third pipeline 530 through the magnetic force of the second magnetic components 180 to achieve the effect of removing the magnetic beads 202. The hollow column 110 has a length of 160 mm and a diameter of 10 mm. The total length of the blind tubes 170 in the radial direction of the hollow column 110 is 5 mm. The thickness of the first magnetic component 130 is 15 mm and the length thereof is 40 mm. A thickness of the second magnetic component 180 is 6 mm and a length thereof is 30 mm.

To be specific, in the embodiment, FIG. 5 schematically illustrates two second magnetic components 180. The two second magnetic components 180 are respectively disposed at two sides of the third pipeline 530. The two second magnetic components 180 are, for example, directly or indirectly bound to two sides of the third pipeline 530, but the disclosure is not limited thereto. The two second magnetic components 180 are, for example, arranged on the upper and lower sides of the third pipeline 530 in a direction Y perpendicular to the extending direction X of the hollow column 110, but the disclosure is not limited thereto. In the embodiment, a material of the second magnetic component 180 is, for example, the same as or similar to the material of the first magnetic component 130, which is not repeated here.

Although the number of the second magnetic components 180 schematically illustrated in the embodiment is two, and the two second magnetic components 180 are respectively disposed at the two sides of the hollow column 110, the disclosure does not limit the number and configuration positions of the second magnetic components 180, as long as the second magnetic components 180 can be disposed at the periphery of the third pipeline 530 and adjacent to the fluid sample inlet end 140 of the cell purification module 100. In some embodiments, the number of the second magnetic component may also be one, which is disposed at one side of the third pipeline (not shown). In some embodiments, the number of the second magnetic components may also be two or more, which are respectively disposed at two or more sides of the third pipeline (not shown). In some embodiments, the number of the second magnetic components may also be two or more, which annularly surround the third pipeline (not shown). In some embodiments, the number of the second magnetic components may also be two, which are all disposed at the same side of the third pipeline (not shown). In some embodiments, the number of the second magnetic component may also be one, and the second magnetic component may be embodied as a ring structure and may annularly surround the third pipeline (not shown).

### EXAMPLES

### Example 1: Tests of the Magnetic Bead Removal Capability of Various Magnetic Component Configurations

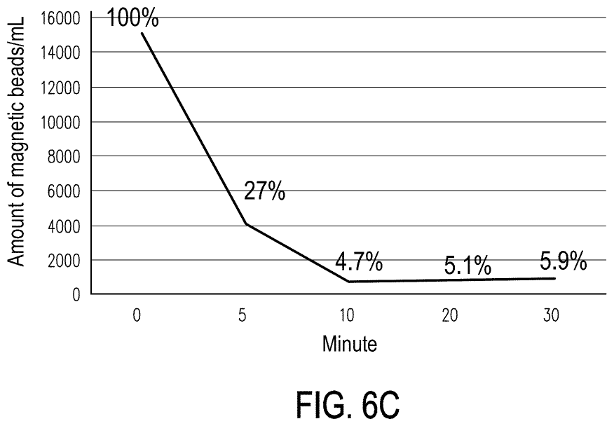

In the embodiment, the cell purification systems containing the cell purification modules as shown in FIGS. 2A, 2B, and 3A and the cell purification system as shown in FIG. 5 are respectively used to remove the magnetic beads from the cellular fluid. The total length of the blind tubes in the radial direction of the hollow column is 6 mm, and a diameter of the hollow column 110 is 10 mm. Then, a flow cytometer is used to measure a concentration of the magnetic beads (i.e., the number of magnetic beads per milliliter) of the cellular fluid in the storage container at a specific time. The specific time includes before purification (i.e., a 0^{th} minute) and a 5^{th}, 10^{th}, 20^{th}, and 30^{th} minutes after the start of purification. Then, a magnetic bead removal ratio is calculated according to an equation: magnetic bead removal ratio (%)=100−magnetic bead remaining ratio (magnetic bead concentration after the purification is started/magnetic bead concentration before the purification)×100%. The results are shown in FIGS. 6A to 6C.

Referring to FIG. 6A, FIG. 6A is a result of using the cell purification system including the cell purification module as shown in FIG. 2A to remove the magnetic beads from the cellular fluid. According to the result shown in FIG. 6A, the magnetic beads remaining ratio at the 5th 10^{th}, 20^{th}, and 30^{th} minutes after the start of purification were about 41%, 22%, 13%, and 11%, respectively. In other words, the magnetic bead removal ratio at the 5^{th}, 10^{th}, 20^{th}, and 30^{th} minutes after the start of purification were approximately 59%, 78%, 87%, and 89%, respectively.

Referring to FIG. 6B, FIG. 6B is a result of using the cell purification system including the cell purification module as shown in FIG. 2B to remove the magnetic beads from the cellular fluid. According to the result shown in FIG. 6B, the magnetic beads remaining ratio at the 5^{th}, 10^{th}, 20^{th}, and 30^{th} minutes after the start of purification were about 30%, 8.7%, 3.7%, and 4.3%, respectively. In other words, the magnetic bead removal ratio at the 5^{th}, 10^{th}, 20^{th}, and 30^{th} minutes after the start of purification were approximately 70%, 91.3%, 96.3%, and 95.7%, respectively.

Referring to FIG. 6C, FIG. 6C is a result of using the cell purification system as shown in FIG. 5 to remove the magnetic beads from the cellular fluid. According to the result in FIG. 6C, the magnetic beads remaining ratio at the 5^{th}, 10^{th}, 20^{th}, and 30^{th} minutes after the start of purification were about 27%, 4.7%, 5.1%, and 5.9%, respectively. In other words, the magnetic bead removal ratio at the 5^{th}, 10^{th}, 20^{th}, and 30^{th} minutes after the start of purification were approximately 73%, 95.3%, 94.9%, and 94.1%, respectively.

### Example 2: Using a Cell Purification System with a Cell Purification Module to Remove Magnetic Beads and to Purify Cells

In the embodiment, the cell purification system containing the cell purification module as shown in FIG. 3A is used to firstly remove the magnetic beads from the cellular fluid and secondly purify the cells in the cellular fluid. Prior to cell purification process, flow cytometer is used to measure the concentration of magnetic beads in the cellular fluid and the ratio of cells containing cell markers. Total cell concentration, viable cell concentration, and extracellular substance concentration will be measured by conventional methods described in the text below. The cell purification system is set up to implement various magnet arrangements, and 100 mL of the cellular fluid containing magnetic beads is injected into the cell purification system through a peristaltic pump to perform continuous cyclic concentration at a flow rate of 150 mL/min. Then, 100 mL of unconcentrated cellular fluid is concentrated to 50 mL. Then, three washing procedures are respectively performed, where 150 mL of phosphate buffered saline (PBS) was added for each washing procedure, and then the 200 mL of solution is concentrated to 50 mL. Thereafter, about 34.5 mL of concentrated cellular fluid is collected. After cell purification process is completed, the flow cytometer is used to measure the concentration of magnetic beads and the ratio of cells containing cell markers. Total cell concentration, viable cell concentration, and extracellular substance concentration will be measured by conventional methods described in the text below. The cell markers include CD3, CD4, CD8, CD14 and CD19, but the disclosure is not limited thereto. The total length of the blind tubes in the radial direction of the hollow column is 6 mm, and the diameter of the hollow column is 10 mm.

Then, a magnetic bead removal ratio, a cell recovery ratio, a cell viability, an extracellular substance removal ratio, and a cell concentration ratio are calculated based on the following equations. Cell recovery ratio (%)=(cell concentration of the cellular fluid after concentration×volume)/(cell concentration of the cellular fluid before concentration×volume)×100%. Cell viability (%)=(concentration of viable cells of the cellular fluid after concentration)/(concentration of total cells of the cellular fluid after concentration)×100%. Extracellular substance removal ratio (%)=100-(concentration of extracellular substances of the cellular fluid after concentration×volume)/(concentration of extracellular substances of the cellular fluid before concentration×volume)×100%. Cell concentration ratio (%)=cell concentration after concentration/cell concentration before concentration×100%. The results are shown in Table 1.

TABLE 1

| | Cell | | Extracellular | Cell |
|---|---|---|---|---|
| Magnetic bead | recovery | Cell | substance | concentration |
| removal ratio | ratio | viability | removal ratio | ratio |
| 99.1% | 95.2% | 94% | 98.2% | 300% |

In the calculation of the extracellular substance removal ratio, for example, the calculation of the human serum albumin (HSA) removal ratio is taken as an example for description. For example, in the cellular fluid before concentration, the concentration of HSA is 188.4 ng/mL, a volume of cellular fluid before concentration is 100 mL, and in the cellular fluid after concentration, the concentration of HSA is 9.8 ng/mL, and the volume of the cellular fluid after concentration is 34.5 mL. Therefore, HSA removal ratio (%)=100−(9.8 ng/mL×34.5 mL)/(188.4 ng/mL×100 mL)×100%=98.2%.

In addition, in the embodiment, the steps for measuring the concentration of HSA are, for example, but not limited to using enzyme-linked immunosorbent assay (ELISA) to analyze the concentration of human serum albumin (HSA) in the cell culture medium before and after concentration, and a reagent group is human serum albumin DuoSet ELISA (R&D systems, DY1455). First, an anti-albumin antibody (served as capture antibody during the capture stage) is immobilized on a 96 well plate by non-covalent adsorption. After the 96 well plate is washed, a culture solution is added to react at room temperature for 2 hours. Thereafter, the unbound material is washed away, and detection is carried out through the anti-albumin antibody bound to biotin (served as detect antibody during the detection stage), and then avidin labeled with horseradish peroxidase (HRP) and enzyme are used for color presentation reaction. Finally, spectrophotometry is used to measure an amount of colored products produced by the reagent. At the same time, serially diluting a standard albumin solution of a known concentration can be made to establish a standard curve, and the standard curve can be incorporated into the ELISA analysis, and the concentration of HSA in the culture medium can be estimated by interpolation.

In addition, in the embodiment, the steps for calculating the concentration of viable cells are, for example, but not limited to utilizing trypan blue exclusion method. Briefly, the trypan blue exclusion method is performed by mixing the cellular fluid with trypan blue in equal volumes and adding a small amount of the mixture (about 20 μl) to an upper groove of a hemocytometer chamber. Then, placing the hemocytometer chamber under a 100λ inverted microscope for observing the cell survival situation is performed. Among the cells, live cells are not stained while dead cells are stained blue. Thereafter, the number of cells per milliliter of cell fluid is obtained by counting the total number of live cells in four large squares on the chamber, determining the average number with dividing by 4, and back-estimating the actual number with multiplying by the dilution factor 10⁴.

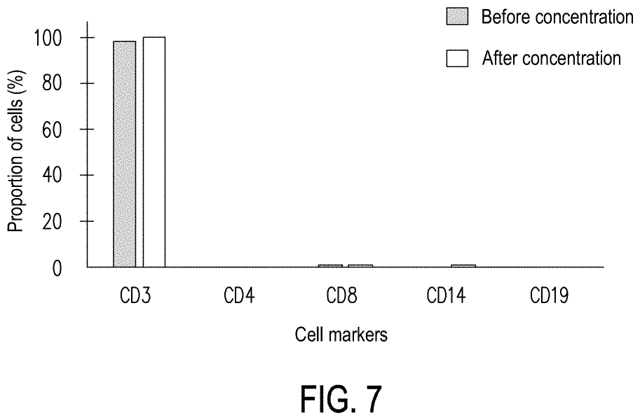

FIG. 7 shows proportions of cells containing specific cell markers in the fluid sample before and after purification. Referring to Table 2 and FIG. 7 concurrently, where Table 2 is a quantification result in FIG. 7.

TABLE 2

| | Proportion of cells containing cell markers (%) | | | | |
|---|---|---|---|---|---|
| | CD3 | CD4 | CD8 | CD14 | CD19 |
| Before | 98.75 | 0.24 | 1.25 | 0.29 | 0.42 |
| purification | | | | | |
| After | 99.90 | 0.45 | 1.32 | 0.99 | 0.32 |
| purification | | | | | |

According to the results in FIG. 7 and Table 2, it can be known that the proportion of cells containing the specific cell markers in the fluid sample before purification is substantially similar to the proportion of cells containing the specific cell markers in the fluid sample after purification. In other words, when the cell purification module and cell purification system of the embodiment are used to remove magnetic beads and purify cells, the specific cell markers on the cell surfaces are not significantly changed, which means that a cell property before and after purification may be maintained and not affected by purification.

In the embodiment, the steps for measuring cell markers are for example, but not limited to each set of cell samples is 10⁵/test, and the antibodies used for analysis are as follows. PE mouse anti-human CD3 (BD), FITC mouse anti human CD4 (BD), PE mouse anti human CD8 (BD), FITC mouse anti human CD14 (BD), FITC mouse anti human CD19 (BD) are incubated for 30 minutes at 4° C. in the dark, and a flow cytometer (FACS Calibur; BD) is used to analyze a cell surface antigen.

In summary, in the cell purification module, the cell purification system, and the operation method thereof of the embodiments of the disclosure, the cell culture medium and the extracellular substances in the cellular fluid may be discharged through the disposition of the pores of the hollow fiber membranes and the filtrate outlet end, so that the cellular fluid may be concentrated and purified. The multiple magnetic beads in the cellular fluid may be adsorbed on the inner walls of the hollow fiber membranes through the disposition of the first magnetic component, so that most of the magnetic beads in the cellular fluid may be removed. Therefore, compared to the related art in which the magnetic bead removal device is used before the cell concentration device (i.e., the magnetic beads are removed first, and then the cells are concentrated), the cell purification system including the cell purification module provided by the disclosure is an integrated and closed system. This integrated and closed system reduces the risk of contamination by concurrently performing both steps of removing the magnetic beads and concentrating the cells, thereby achieving the effects of simplifying the purification process, saving time and improving purification efficiency simultaneously.

Although the disclosure has been described with reference to the above-mentioned embodiments, it is not intended to be exhaustive or to limit the disclosure to the precise form or to exemplary embodiments disclosed. It is apparent to one of ordinary skill in the art that modifications to the described embodiments may be made without departing from the spirit and the scope of the disclosure. Accordingly, the scope of the disclosure is defined by the claims appended hereto and their equivalents in which all terms are meant in their broadest reasonable sense unless otherwise indicated.

What is claimed is:

1. A cell purification module, configured to purify a plurality of cells from a fluid sample, comprising:
a hollow column, having a first opening, a second opening opposite to the first opening, and an accommodating space connecting the first opening and the second opening;
a plurality of hollow fiber membranes, disposed in the accommodating space, and each of the hollow fiber membranes has a plurality of pores;
at least one first magnetic component, disposed at a periphery of the hollow column;
a fluid sample inlet end, disposed at one end of the hollow column; and
a fluid sample outlet end, disposed at another end of the hollow column,
wherein the hollow column has an axial direction extending in a direction of an axis of the hollow column, a radial direction extending in a cross-sectional radius direction of the hollow column and perpendicular to the axial direction of the hollow column, and a circumferential direction surrounding the axis of the hollow column, the plurality of hollow fiber membranes extend in the axial direction of the hollow column, and the plurality of hollow fiber membranes are arranged in the radial direction of the hollow column.

2. The cell purification module according to claim 1, wherein the at least one first magnetic component comprises one or more first magnetic components disposed at at least one side along the circumferential direction of the hollow column.

3. The cell purification module according to claim 1, wherein the at least one first magnetic component comprises one or more first magnetic components annularly surrounding the hollow column.

4. The cell purification module according to claim 1, further comprising:
at least one filtrate outlet end, disposed on the hollow column, so that a filtrate flowing out from the plurality of pores flows out through the filtrate outlet end.

5. The cell purification module according to claim 1, wherein the fluid sample comprises at least one cellular fluid and a plurality of magnetic beads, and the cellular fluid comprises at least a plurality of cells and a cell culture medium.

6. The cell purification module according to claim 1, further comprising:
a plurality of blind tubes, disposed in the accommodating space, wherein the plurality of blind tubes extend in the axial direction of the hollow column and are arranged in the radial direction of the hollow column, and the plurality of blind tubes are surrounded by the plurality of hollow fiber membranes.

7. The cell purification module according to claim 6, wherein the plurality of blind tubes and the plurality of hollow fiber membranes are all annularly arranged around the axis of the hollow column, and a ratio of a total length of the plurality of blind tubes in the radial direction of the hollow column to a diameter of the hollow column is 1:10 to 8:10.

8. The cell purification module according to claim 5, wherein a size of the plurality of cells is larger than a pore diameter of the plurality of pores of the plurality of hollow fiber membranes.

9. A cell purification system, comprising:
a cell purification module, configured to purify a plurality of cells from a fluid sample, comprising:
a hollow column, having a first opening, a second opening opposite to the first opening, and an accommodating space connecting the first opening and the second opening;
a plurality of hollow fiber membranes, disposed in the accommodating space, and each of the hollow fiber membranes has a plurality of pores;
at least one first magnetic component, disposed at a periphery of the hollow column;
a fluid sample inlet end, disposed at one end of the hollow column; and
a fluid sample outlet end, disposed at another end of the hollow column,
wherein the hollow column has an axial direction extending in a direction of an axis of the hollow column, a radial direction extending in a cross-sectional radius direction of the hollow column and perpendicular to the axial direction of the hollow column, and a circumferential direction surrounding the axis of the hollow column, the plurality of hollow fiber membranes extend in the axial direction of the hollow column, and the plurality of hollow fiber membranes are arranged in the radial direction of the hollow column;
a storage container, configured to store the fluid sample, and connected to the fluid sample outlet end of the cell purification module through a first pipeline; and
a peristaltic pump, configured to push the fluid sample to flow, wherein the peristaltic pump is respectively connected to the storage container and the fluid sample inlet end of the cell purification module through a second pipeline and a third pipeline.

10. The cell purification system according to claim 9, further comprising:
at least one second magnetic component, disposed at a periphery of the third pipeline and adjacent to the fluid sample inlet end.

11. The cell purification system according to claim 9, wherein the at least one second magnetic component comprises one or more second magnetic components disposed at at least one side of the third pipeline.

12. The cell purification system according to claim 9, wherein the at least one second magnetic component comprises one or more second magnetic components annularly surrounding the third pipeline.

13. An operation method of a cell purification system, comprising the following steps:
(a) providing a cell purification system, comprising:
a cell purification module, configured to purify a plurality of cells from a fluid sample, comprising:
a hollow column, having a first opening, a second opening opposite to the first opening, and an accommodating space connecting the first opening and the second opening;
a plurality of hollow fiber membranes, disposed in the accommodating space, and each of the hollow fiber membranes has a plurality of pores;
at least one first magnetic component, disposed at a periphery of the hollow column;
a fluid sample inlet end, disposed at one end of the hollow column; and
a fluid sample outlet end, disposed at another end of the hollow column,
wherein the hollow column has an axial direction extending in a direction of an axis of the hollow column, a radial direction extending in a cross-sectional radius direction of the hollow column and perpendicular to the axial direction of the hollow column, and a circumferential direction surrounding the axis of the hollow column, the plurality of hollow fiber membranes extend in the axial direction of the hollow column, and the plurality of hollow fiber membranes are arranged in the radial direction of the hollow column;
a storage container, configured to store the fluid sample, and connected to the fluid sample outlet end of the cell purification module through a first pipeline; and
a peristaltic pump, configured to push the fluid sample to flow, wherein the peristaltic pump is respectively connected to the storage container and the fluid sample inlet end of the cell purification module through a second pipeline and a third pipeline;
(b) using the peristaltic pump to output the fluid sample in the storage container to the peristaltic pump through the second pipeline;
(c) using the peristaltic pump to input the fluid sample into the cell purification module through the third pipeline and the fluid sample inlet end;
(d) using the peristaltic pump to enable a filtrate to flow out through the at least one filtrate outlet end, so as to concentrate the fluid sample and output the concentrated fluid sample through the fluid sample outlet end;
(e) using the peristaltic pump to re-input the concentrated fluid sample outputted from the cell purification module into the storage container through the first pipeline; and
(f) repeating steps (b) to (e).

14. The operation method of the cell purification system according to claim 13, further comprising:
adding a washing solution before performing Step (f).
